(19) United States
(12) Patent Application Publication
(10) Pub. No.: US 2020/0258641 A1
(43) Pub. Date: Aug. 13, 2020

(54) WARNING DEVICE, WARNING METHOD, AND WARNING PROGRAM

(71) Applicant: FUJIFILM Corporation, Tokyo (JP)

(72) Inventors: Keigo NAKAMURA, Tokyo (JP); Noriaki IDA, Tokyo (JP)

(73) Assignee: FUJIFILM Corporation, Tokyo (JP)

(21) Appl. No.: 16/860,035

(22) Filed: Apr. 27, 2020

Related U.S. Application Data
(63) Continuation of application No. PCT/JP2018/043277, filed on Nov. 22, 2018.

(30) Foreign Application Priority Data
Dec. 1, 2017 (JP) 2017-232053

(51) Int. Cl. G16H 80/00; G08B 21/18; G16H 30/40; G16H 50/20; G06F 9/445

(52) U.S. Cl. CPC G16H 80/00; G08B 21/18; G06F 9/445; G16H 50/20; G16H 30/40

(57) ABSTRACT

An object of the present invention is to provide a warning device, a warning method, and a warning program which monitor an operation of a user and do not neglect interrupted interpretation. In a state in which an operation input is executable during execution of an interpretation support program for supporting interpretation of a medical image, a warning is issued in a case where an operation of resuming the interrupted interpretation is not detected within a predetermined time from a time at which an operation of interrupting the interpretation is detected.

### CROSS-REFERENCE TO RELATED APPLICATIONS

This application is a Continuation of PCT International Application No. PCT/JP2018/043277 filed on Nov. 22, 2018, which claims priority under 35 U.S.C § 119(a) to Japanese Patent Application No. 2017-232053 filed on Dec. 1, 2017. Each of the above application(s) is hereby expressly incorporated by reference, in its entirety, into the present application.

### BACKGROUND OF THE INVENTION

### 1. Field of the Invention

The present invention relates to a warning device, a warning method, and a non-transitory computer readable recording medium storing a warning program for supporting diagnosis in medical treatment.

### 2. Description of the Related Art

In recent years, imaging devices including Computed Tomography (CT), magnetic resonance imaging (MM), and positron emission tomography (PET) have been widely used, and have been adopted in many hospitals. Image diagnosis of medical images captured by these imaging device for capturing has been performed by an interpretation specialist (hereinafter, referred to as a radiologist) by using an interpretation terminal, and has been used for various diagnoses.

The radiologist interprets a plurality of interpretation requests listed on the interpretation terminal, and creates an interpretation report (a report describing the results of diagnosis or determination and a treatment policy). At this time, diagnosis of a case to be interpreted cannot be diagnosed by the radiologist alone, and may consult another doctor. Alternatively, consultations for other cases may be requested from other doctors. Such a consultation may be performed by exchanging messages with a doctor at a location separated from the terminal device via a network, and the interpretation is temporarily interrupted until the exchange with a consultation partner is ended. Apart from that, in a case where the interpretation is performed in order from the requested list, there may be another request which is not on the request list, is urgent, and needs to be preferentially interpreted. In such a case, the current interpretation is interrupted, and interpretation of another urgent case is started.

Since the radiologist interprets a plurality of medical images for a long time, the radiologist may leave the seat for a long time or perform another work in the middle of the interpretation. In a case where the medical image to be interpreted is displayed on the terminal device for such a long time, the medical image cannot be used by other devices other than the terminal device during the interpretation. Therefore, JP2009-082182A discloses a method of immediately starting measurement of a time in a case where a medical image is displayed on a display and displaying a warning message on a display unit in a case where it is determined that an interpretation time elapses by a set display time.

The radiologist may be tired and unable to concentrate on the interpretation by conducting the interpretation over a long time. Therefore, JP2005-348936A discloses a method of displaying a medical image on a display unit and starting measurement of a time and displaying a warning message on the display unit in a case where it is determined that an interpretation time elapses by a set display time in order to perform a test for determining a degree of tiredness of the radiologist.

### SUMMARY OF THE INVENTION

As described above, the radiologist often interrupts the current interpretation work, and starts another interpretation. However, in a case where the radiologist requests the consultant with another doctor via a network and the consultation partner did not respond to this request, or in a case where the interpretation of the urgent case is requested and it took a long time to interpret this urgent case, the data being interpreted is neglected for a long time before the interruption, and thus, the response for the patient may be delayed.

An object of the present invention is to provide a warning device, a warning method, and a non-transitory computer readable recording medium storing a warning program which prevent interrupted interpretation from being neglected by monitoring an operation of a doctor.

A warning device according to the present invention comprises a detection unit that detects an operation of interrupting interpretation in a state in which an operation input is executable during execution of an interpretation support program for supporting interpretation of a medical image, and a warning unit that issues a warning in a case where an operation of resuming the interrupted interpretation is not detected within a predetermined time from a time at which the interruption operation is detected.

A warning method performed by a warning device according to the present invention comprises a detection step of detecting an operation of interrupting interpretation in a state in which an operation input is executable during execution of an interpretation support program for supporting interpretation of a medical image, and a warning step of issuing a warning in a case where an operation of resuming the interrupted interpretation is not detected within a predetermined time from a time at which the interruption operation is detected.

A non-transitory computer readable recording medium storing a warning program according to the present invention causes a computer to execute a detection step of detecting an operation of interrupting interpretation in a state in which an operation input is executable during execution of an interpretation support program for supporting interpretation of a medical image, and a warning step of issuing a warning in a case where an operation of resuming the interrupted interpretation is not detected within a predetermined time from a time at which the interruption operation is detected.

The “state in which the operation input is executable” refers to a state in which the operation is executable on an interface provided by the interpretation support program, and refers to a state in which the interpretation report can be input.

The interruption operation may be an operation of starting execution of another program other than the interpretation support program.

The other program may be a program for transmitting and receiving a message related to a conference of the medical image to and from another terminal device.

The “transmitting and receiving the message related to the conference” refers to the exchange of the message for electronically conducting the conference between a plurality of users, and may be performed by using a mechanism such as a chat, an electronic conference or an electronic bulletin board.

The interruption operation may be an operation of switching an image to a medical image other than the medical image being interpreted.

The operation of switching the image may be an operation of switching the image to the medical image for which the interpretation is interrupted during execution of interpretation of a predetermined medical image or an operation of switching the image to a new medical image for which the interpretation is started during the execution of the interpretation of the predetermined medical image.

Another warning device according to the present invention comprises a memory that stores a command to be executed by a computer, and a processor configured to execute the stored command. The processor executes a detection step of detecting an operation of interrupting interpretation in a state in which an operation input is executable during execution of an interpretation support program for supporting interpretation of a medical image, and a warning step of issuing a warning in a case where an operation of resuming the interrupted interpretation is not detected within a predetermined time from a time at which the interruption operation is detected.

According to the present invention, during the interpretation of the medical image, the interpreted interpretation is neglected for a long time by issuing the warning in a case where the operation of resuming the interrupted interpretation is not detected within the predetermined time from the time at which the operation of interrupting the interpretation is detected. It is possible to prevent the interpretation of the patient from being neglected.

### BRIEF DESCRIPTION OF THE DRAWINGS

FIG. 1 is a diagram showing a schematic configuration of a medical information system.

FIG. 2 is a diagram showing a schematic configuration of a warning device and a terminal device according to a first embodiment of the present invention.

FIG. 3 is a state transition diagram of a program of the present invention.

FIG. 4A is a flowchart (part 1) showing a flow of processing of interpretation according to the first embodiment of the present invention.

FIG. 4B is a flowchart (part 2) showing the flow of the processing of the interpretation according to the first embodiment of the present invention.

FIG. 5 is an example of a screen at the time of interpreting a medical image.

FIG. 6 is an example of a screen at the time of starting interpretation of another medical image by switching the medical image being interpreted.

FIG. 7 shows an example of a screen on which a warning message is displayed.

FIG. 8 is an example of a screen in a case where a conference is being performed by switching a screen during the interpretation.

FIG. 9 is a diagram showing a schematic configuration of a warning device according to a second embodiment of the present invention.

FIG. 10A is a flowchart (part 1) showing a flow of processing of interpretation according to the second embodiment of the present invention.

FIG. 10B is a flowchart (part 2) showing the flow of the processing of the interpretation according to the second embodiment of the present invention.

FIG. 11 shows an example of a state transition table.

### DESCRIPTION OF THE PREFERRED EMBODIMENTS

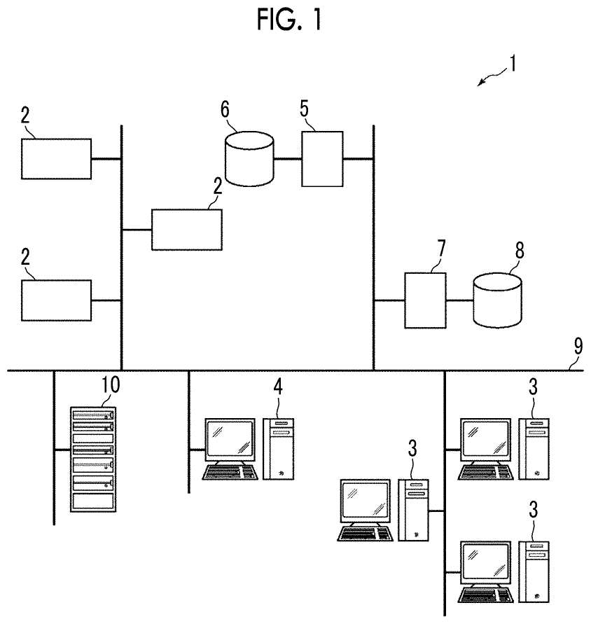

Hereinafter, a medical support system according to a first embodiment of the present invention will be described with reference to the drawings. FIG. 1 is a block diagram showing a schematic configuration of a medical information system including a warning system according to the present embodiment.

The medical information system 1 shown in FIG. 1 is a system configured to capture an inspection target portion of a subject based on an inspection order from a doctor in a clinical department using a known ordering system, store a medical image acquired by the capturing, perform medical interpretation of the medical image by a radiologist and creation of an interpretation report, and perform browsing of the interpretation report by the doctor in the clinical department as a requesting party and detailed observation of the medical image to be interpreted. As shown in FIG. 1, the medical information system 1 is configured such that a plurality of modalities (imaging devices) 2, a plurality of interpretation workstations 3 (hereinafter, referred to as interpretation WS 3) which is interpretation terminals, a clinical department workstation 4 (hereinafter, referred to as a clinical department WS 4), an image server 5, an image database 6, an interpretation report server 7, an interpretation report database 8, and a warning device 10 according to the present embodiment are connected to be able to communicate with each other via a network 9.

Each device is a computer on which an application program for functioning as a component of the medical information system 1 is installed. The application program is distributed while being recorded on a recording medium such as a Digital Versatile Disc (DVD) or a Compact Disc Read Only Memory (CD-ROM), and is installed on the computer from the recording medium. Alternatively, the application program is stored in a storage device of a server computer connected to a network or a network storage in a state of being accessible from the outside, and is downloaded and installed on the computer according to a request.

The modality 2 is a device that generates a medical image representing a diagnosis target portion by capturing the diagnosis target portion of the subject. Specifically, the modality is a CT device, an MM device, a Computed Radiography (CR) device, a PET device, or an ultrasonic device. The captured medical images are stored in a storage format conforming to Digital Imaging and Communication in Medicine (DICOM) standard according to a communication standard, and are transmitted to the image server 5 via the network 9.

The interpretation WS 3 is a computer that is used by the radiologist of the medical image for the interpretation of the medical image and the creation of the interpretation report, and includes a processing device, a high-definition display, and input devices such as a keyboard and a mouse. In the interpretation WS 3, various processing such as the browsing request for the medical image to the image server 5, various image processing on the medical image received from the image server 5, the display of the medical image, support of the creation of the interpretation report, the registration request and the browsing request for the interpretation report to the interpretation report server 7, and the display of the interpretation report received from the interpretation report server 7 is performed by executing the software program for performing each processing. A separate image processing server may be connected to the network 9 without performing various image processing in the interpretation WS 3, and the image processing server may perform the various image processing and the analysis of the medical image according to the processing request from the interpretation WS 3.

The clinical department WS 4 is a computer to be used by the doctor in the clinical department for the detailed observation of the image, browsing of the interpretation report, and creation of an electronic medical record, and includes a processing device, a high-definition display, and input devices such as a keyboard and a mouse. In the clinical department WS 4, each processing of a browsing request for an image to the image server 5, display of an image received from the image server 5, automatic detection or emphasis display of a lesion-like portion in the image, and a browsing request of an interpretation report to the interpretation report server 7, and display of the interpretation report received from the interpretation report server 7 is performed by executing a software program for each processing.

The image server 5 is a general-purpose computer on which a software program for providing a function of a database management system (DBMS) having a relatively high processing capability is installed. The image server 5 has a large-capacity storage including the image database 6. This storage may be a large-capacity hard disk device connected to the image server 5 by a data bus, or may be a disk device connected to a Network Attached Storage (NAS) and a Storage Area Network (SAN) connected to the network 9. In a case where a registration request for the medical image from the modality 2 is received, the image server 5 prepares the medical image in a database format, and registers the medical image in the image database 6.

In the image database 6, image data of the medical image acquired in the modality 2 and accessory information are registered. The accessory information may include, for example, an image ID for identifying an individual medical image, a patient identification (ID) for identifying a subject, an inspection ID for identifying an inspection, a unique identification (UID) assigned to each medical image, an inspection date and an inspection time at which the medical image is generated, a type of the modality used in the inspection for acquiring the medical image, patient information such as a patient name, an age, and a gender, an inspection portion (imaging portion), imaging information (imaging protocol, imaging sequence, imaging method, imaging condition, and use of contrast agent), and information of a series number or a collection number in a case where a plurality of medical images is acquired in one inspection.

In a case where the browsing request from the interpretation WS 3 is received via the network 9, the image server 5 searches for the medical image registered in the image database 6, and transmits the extracted medical image to the interpretation WS 3 as the requesting party.

The interpretation report server 7 incorporates a software program for providing a function of a database management system to a general-purpose computer. In a case where a registration request for the interpretation report from the interpretation WS 3 is received, the interpretation report server 7 prepares the interpretation report in a database format, and registers the interpretation report in the interpretation report database 8.

For example, an interpretation report in which information such as an image ID for identifying the medical image to be interpreted, a radiologist ID for identifying an image radiologist who performs interpretation, a lesion name, positional information of a lesion, a medical opinion, and a degree of certainty of the medical opinion is recorded are recorded in the interpretation report database 8.

The network 9 is a wired or wireless local area network for connecting various devices in a hospital. In a case where the interpretation WS 3 is installed in another hospital or clinic, the network 9 may have a configuration in which local area networks of the hospitals are connected to each other via the Internet or a dedicated line. In any case, it is preferable that the network 9 has a configuration capable of realizing high-speed transfer of the medical images such as an optical network.

The warning system according to the present embodiment includes the warning device 10 and a terminal device.

The warning device 10 is a general-purpose computer, and includes a known hardware components such as a Central Processing Unit (CPU) 11, a memory (main memory) 12, a storage (auxiliary memory) 13, an input and output interface (not shown), a communication interface (not shown), and a data bus (not shown).

A known operation system is installed. The storage includes a hard disk or a Solid State Drive (SSD). A Graphics Processing Unit (GPU) may be provided in the computer as needed.

Alternatively, a warning program may be stored in a storage device of a computer connected to a network or a network storage in a state of being accessible from the outside, and may be downloaded and installed on the computer according to a request from the outside. Alternatively, the warning program is distributed while being recorded on a recording medium such as a DVD or a CD-ROM, and is installed on the computer from the recording medium.

In a case where this computer is executed, the warning program is stored in the memory 12. The CPU 11 executes these processing according to the program stored in the memory 12, and thus, the computer functions as the warning device 10 (hereinafter, referred to as a warning server 10).

Meanwhile, in the present embodiment, the interpretation WS 3 functions as a terminal device of the warning system. Hereinafter, the interpretation WS 3 will be described as the terminal device 3.

The terminal device 3 is a computer having a known hardware configuration such as a CPU 31, a memory 32, a storage (not shown), an input and output interface (not shown), a communication interface (not shown), and a data bus (not shown). A display 36 and an input device 37 such as a keyboard and a mouse are connected. In a case where this computer is executed, a terminal device program is stored in the memory 32. The CPU 31 executes these processing according to the program stored in the memory 32, and thus, the computer functions as the terminal device.

A plurality of application programs is installed on the terminal device 3, and an interpretation support program P is included in the installed application programs (hereinafter, simply referred to as programs). The interpretation support program P is a program used for interpreting the medical image as the diagnosis target, and is a program that has a function of displaying the medical image as the diagnosis target and a function of creating the interpretation report by inputting an interpretation result, and stores the medical image and the interpretation report in the interpretation report database 8 by preparing the medical image and the interpretation report in a predetermined format in a case where the input of the interpretation report is ended.

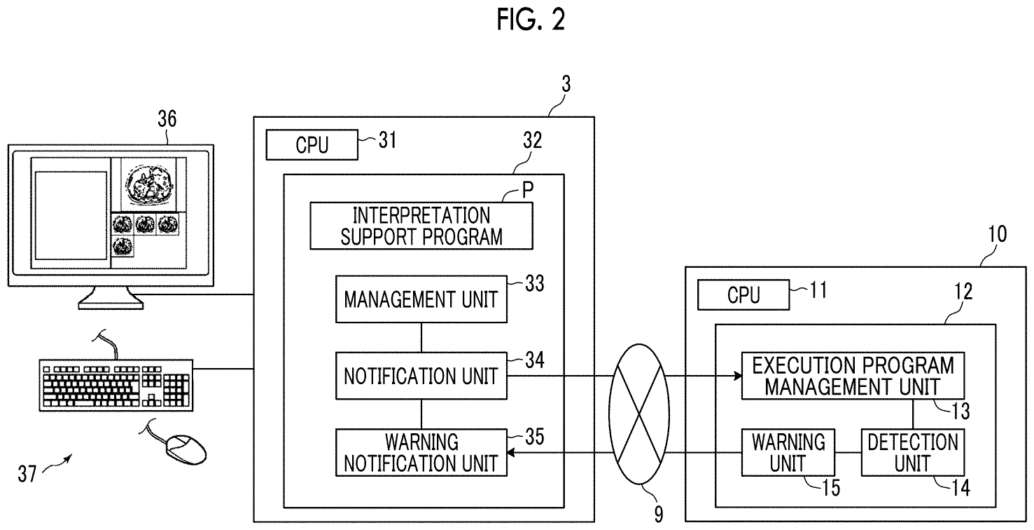

First, a function of the warning server 10 according to the first embodiment will be described. As shown in FIG. 2, the warning server 10 according to the first embodiment functions as an execution program management unit 13, a detection unit 14, and a warning unit 15.

The execution program management unit 13 manages states of all the programs installed on the terminal device 3. The states of the programs include the in-execution of the program, the end of the program, and a state in which a display window for each program is active or inactive. Specifically, information on the state of each program is received from the terminal device 3, and the received information is recorded in a state transition table. The in-execution of the program, the end of the program, the active state, and the inactive state are recorded for each program in the state transition table.

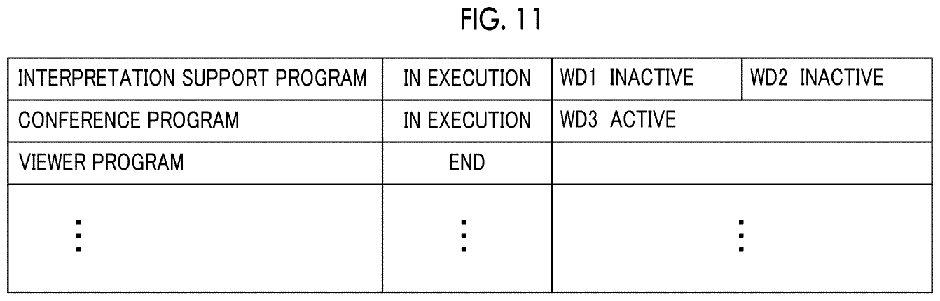

FIG. 11 shows an example of the state transition table. For each program installed on the terminal device 3, whether the program is in execution or is ended is recorded, and whether the display window of each program is active or inactive is further recorded in the state transition table. In FIG. 11, the interpretation support program P and a conference program are in execution, and a viewer program is ended. Two display windows WD1 and WD2 are opened in the interpretation support program P, and WD1 and WD2 are inactive. Meanwhile, the display window WD3 of the conference program is active.

In a case where the interpretation support program P is executed and an operation input such as an interpretation report is executable, the detection unit 14 detects that an operation of interrupting the interpretation is performed on the terminal device 3. For example, the state of the program recorded in the state transition table is checked whenever a notification of the state of the program is received from the terminal device 13, and whether the operation of interrupting the interpretation is performed on the terminal device 3 is determined based on the state of the program. Alternatively, it may be periodically determined whether or not the operation of interrupting the interpretation is performed on the terminal device 3 based on the state of the program. The state in which the operation input is executable refers to a state in which an input for creating the interpretation report for the medical image to be interpreted is executable, and is a state in which the display window for inputting the interpretation report is active. Meanwhile, the state in which the interpretation is interrupted refers to a state in which the interpretation report cannot be input, and is a state in which the display window for inputting the interpretation report is inactive. Therefore, a case where the display window of the medical image to be interpreted is changed from the active state to the inactive state can be detected as the interruption of the interpretation.

The interpretation is interrupted in a case where the medical image being currently interpreted is switched to another medical image while executing the interpretation support program P on the terminal device 3 or another program is executed while executing the interpretation support program P on the terminal device 3. Anther program includes a program for transmitting and receiving messages to and from a terminal device of another doctor, for example, to hold a conference on data of the diagnosis target such as the medical image between two or more doctors. Such a conference support program desirably has a form in which communication is performed in real time such as a chat, an electronic conference, or a bulletin board.

In the detection unit 14, the interpretation support program P detects that an operation of resuming the interpretation from the interruption of the interpretation is performed on the terminal device 3. For example, the state of the program recorded in the state transition table is periodically checked whenever a notification of the state of the program is received from the terminal device 13, and whether or not the interpretation program in a state in which the interpretation is interrupted enters a state in which the operation input is executable is determined. A case where the display window of the interrupted interpretation program is active can be detected as the resumption of the interpretation.

The interpretation is resumed in a case where a user selects and activates the display window of the medical image for which the interpretation is interrupted on the terminal device 3, in a case where the interpretation of another medical image being interpreted is ended and the display window of another medical image is ended, or a case where the execution of the program is ended.

In a case where the operation of resuming the interrupted interpretation is not detected within a predetermined time from a time at which the operation to be interrupted is detected, the warning unit 15 transmits a notification of a warning to the terminal device 3. In a case where a plurality of medical images is to be interpreted, the interruption of the interpretation is detected for each medical image.

Next, a function of the terminal device 3 according to the first embodiment will be described. As shown in FIG. 2, the terminal device 3 according to the first embodiment functions as a management unit 33, a notification unit 34, and a warning notification unit 35.

The management unit 33 constantly manages the states of all the programs installed on the terminal device 30.

The notification unit 34 notifies the warning server 10 of the state of the program managed by the management unit 33. The notification is performed at a predetermined interval, for example. Alternatively, the state of the program may be notified to the warning server 10 whenever the state of the program is changed. The change in the state of the program includes a change in the state caused by information received from the outside such as a push notification received from an external device in addition to a change in the state caused by the operation of the user of the terminal device 3.

In a case where the notification of the warning is received from the warning server 10, the warning notification unit 35 notifies the warning. Specifically, the warning is performed by displaying a message on a screen of the display 36. Alternatively, a mark indicating the warning may be displayed on the screen, or a warning sound may be sounded.

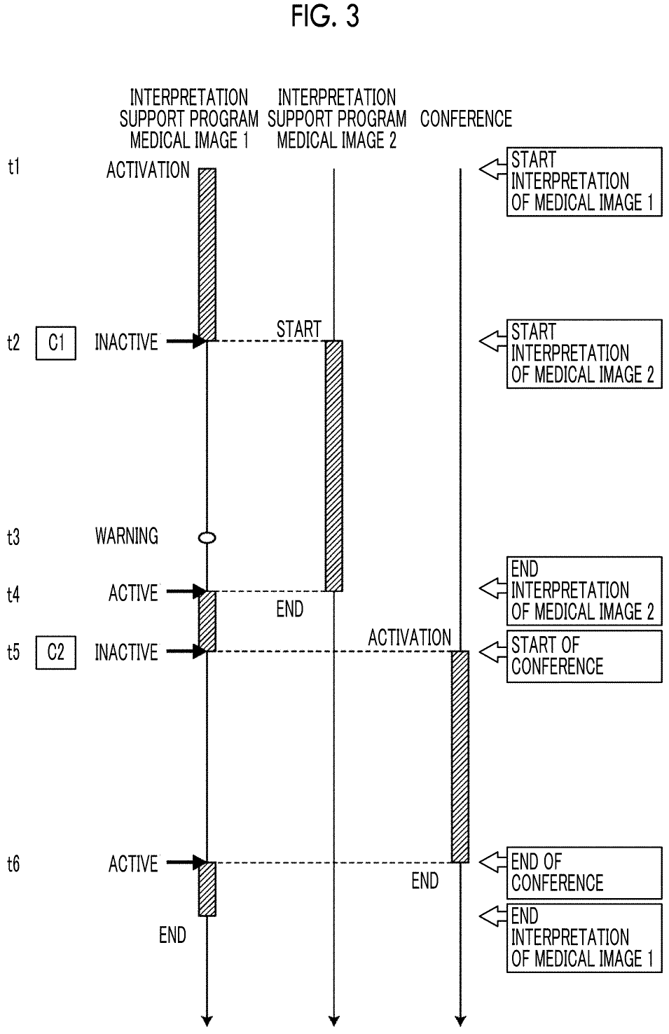

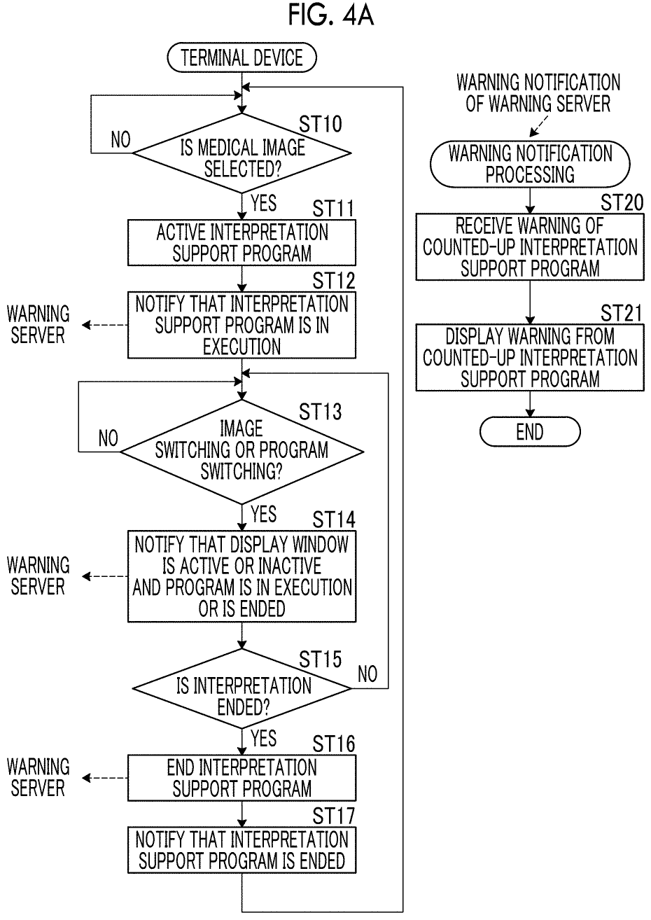

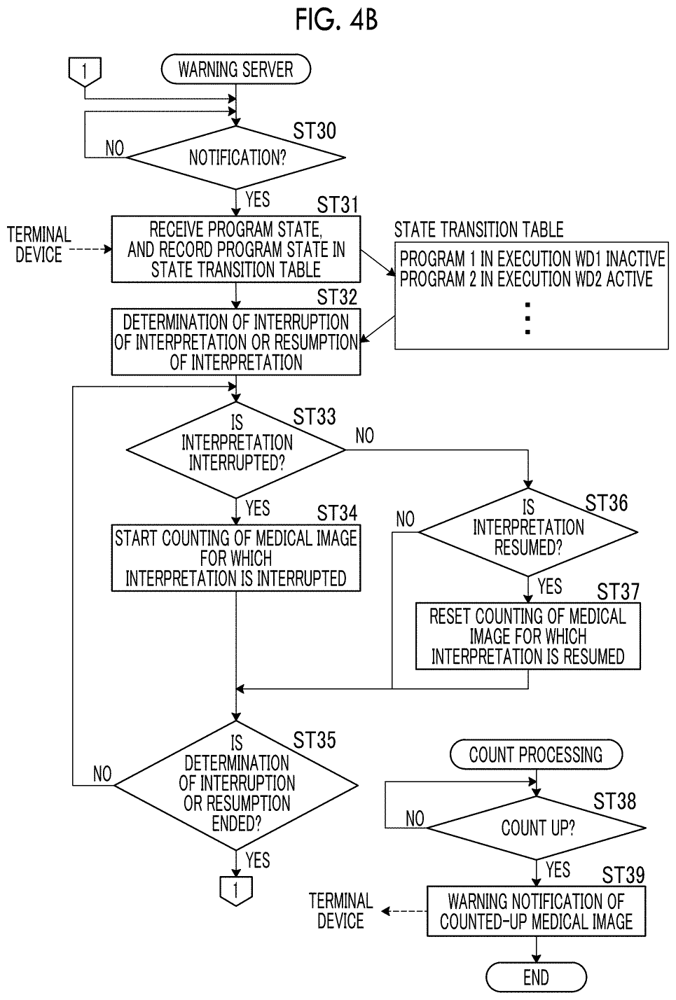

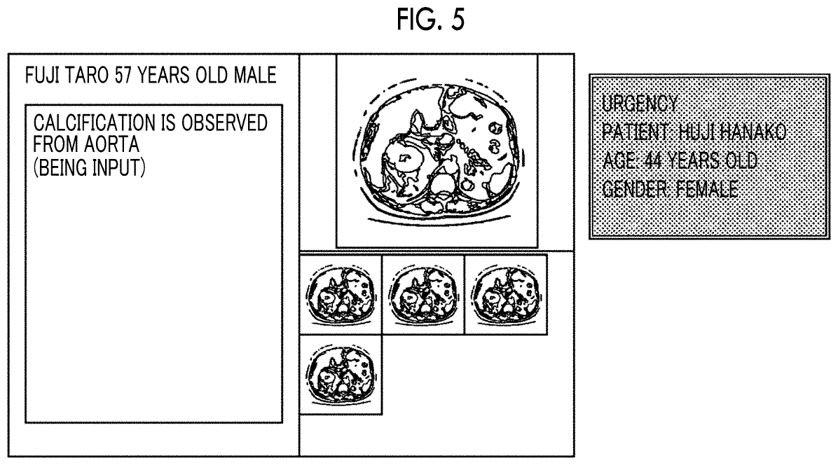

A method of detecting the operations for interrupting the interpretation and resuming the interpretation by using an example of a display screen of the terminal device of FIGS. 5 to 8 according to a state transition diagram of the program in FIG. 3 and flowcharts of FIGS. 4A and 4B will be described. In FIG. 3, a vertical axis is a time axis.

The interpretation is interrupted in a case where the medical image to be interpreted is switched to another medical image during the execution of the interpretation support program, or a case where the program is switched and another program is executed during the execution of the interpretation support program.

First, a case where the medical image to be interpreted is switched to another medical image during the execution of the interpretation support program, that is, a case where the interpretation of the medical image being interpreted is interrupted and the interpretation of a new medical image is started, or a case where the interpretation of the medical image for which the interpretation is interrupted is resumed will be described.

FIG. 4A shows a flow of processing of the warning server 10, and FIG. 4B shows a flow of processing of the terminal device 3. First, a case in which the interpretation of the image being interpreted is interrupted and the interpretation of another image is started on the interpretation support program will be described.

In a case where an interpretation target list is displayed on the terminal device 3 and the user selects a medical image 1 to be interpreted, the determination in step ST10 is positive, and the interpretation support program P is activated from time t1 as shown in FIG. 3 (step ST11). The interpretation support program P acquires the selected medical image 1 to be interpreted from the image server 5, and displays the medical image 1 on the display 36. For example, as shown in FIG. 5, the medical image 1 (an image of patient: Fuji Taro, age: 57 years old, gender: male) is displayed on a right side of the display window, and a report input column is displayed on a left side. The management unit 33 of the terminal device 3 notifies the warning server 10 that the interpretation support program P is in execution on the medical image 1 and that the display window of the medical image 1 is in an active state (step ST12). In a case where the notification is received, the determination of step ST30 is positive, and the execution program management unit 13 of the warning server 10 records the received state of the program in the state transition table (step ST31). In the state transition table, the number of the image active and the number of the image inactive for the interpretation support program are stored according to the notification from the terminal are stored, and a counter for the active image and a counter for the inactive image are stored.

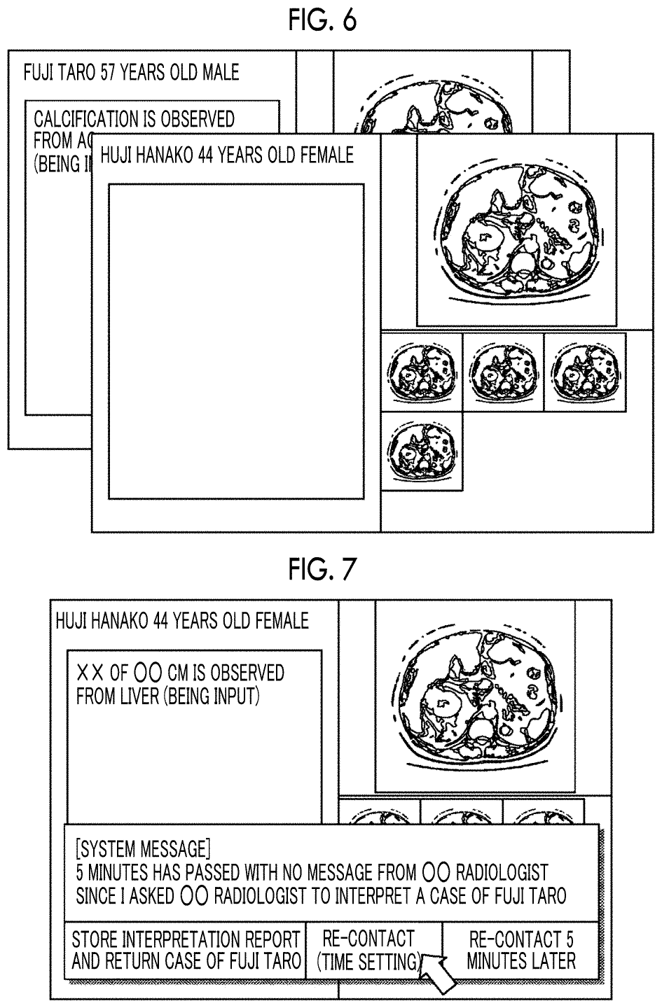

For example, as shown in an upper right frame of FIG. 5, in a case where an urgently interpreted case (patient: Fuji Hanako, age: 44 years old, gender, female) is displayed while the medical image 1 is being interpreted on the terminal device 3, the user returns to the interpretation list, and selects an urgent case. In a case where the urgent case is selected, since the interpretation support program P acquires a medical image 2 (an image of Fuji Hanako) of the selected case from the image server 5, switches from the medical image 1 to the medical image 2, and displays the switched medical image on the display 36, step ST13 is positive. As shown in FIG. 3, from time t2, the display window of the medical image 2 is active, and the display window of the medical image 1 is inactive. For example, an interpretation screen of the patient “Fuji Hanako” is displayed on a front surface and an interpretation screen of the patient “Fuji Taro” is displayed on a rear surface on the display 36 as shown in FIG. 6. The management unit 33 of the terminal device 3 notifies the warning server 10 that the interpretation support program P for the medical image 2 is being executed, that the display window for the medical image 2 is in an active state, and that the display window for the medical image 1 is in an inactive state (step ST14). In a case where the notification is ended, it is determined in step ST15 whether or not the interpretation is ended due to the end of the interpretation support program. The processing returns to step ST13 until the interpretation support program is ended, and is in a waiting state until the switching of the next image or the switching of the program occurs. Steps ST13 and ST14 are repeated.

In a case where the notification is received from the terminal device 3, step ST30 is positive, and the execution program management unit 13 of the warning server 10 records the received state of the program in the state transition table as described above (step ST31). The detection unit 14 of the warning server 10 determines whether the interpretation of the medical image 1 is interrupted or the interpretation is resumed (or started) based on the information of the state transition table (steps ST33 and ST36). In a case where the case is switched to the urgent case, since the display window of the medical image 1 is inactive and it is determined that the interpretation of the medical image 1 is interrupted, step ST33 is positive, and the counting of a counter C1 of the medical image 1 is started from time t2 of FIG. 3 (step ST34). The processing returns from step ST35 to step ST33, and the determination of whether the interruption of the interpretation of the medical image 2 or the resumption (or start) of the interpretation is performed. Since the interpretation of the medical image 2 to be newly interpreted is started, the determination of step ST33 is negative, the determination of step ST36 is positive, and a counter for the medical image 2 is maintained in a reset state (step ST37). Similarly in a case where the interpretation of the interrupted medical image is resumed, a counter of the medical image for which the interpretation is interrupted is also reset before the counter of the medical image for which the interpretation is interrupted is interrupted. In a case where the determination of the interruption of the interpretation or the resumption (or the start) of the interpretation has is completed for all of the active medical image and the inactive medical image being interpreted, the processing returns from step ST35 to step ST30, and is in a waiting state until the next notification is received from the terminal device 3.

Meanwhile, the counter C1 which starts counting is periodically checked by count processing until the counter counts up (step ST38). Since the counter C1 counts up at time t3 elapsed by a predetermined time, for example, five minutes after the interpretation of the medical image 1 is interrupted, that is, the medical image is inactive, step ST38 is positive, and the warning unit 15 of the warning server 10 notifies the terminal device 3 of the warning (step ST39).

In a case where the warning notification unit 35 of the terminal device 3 receives the notification of the warning from the warning server 10 (step ST20), a message of, for example, “5 minutes has passed with no message from OO radiologist since I asked OO radiologist to interpret a case of Fuji Taro” is displayed on the display 36 as shown in FIG. 7 (step ST21). In a case where the user continues to interpret the medical image 2, the user presses a button of “re-contact” to continue the interpretation of the image, and in a case where the interpretation of the medical image 2 is ended, the user presses a button of “store interpretation report and return case of Fuji Taro”. In response to this operation, the interpretation support program P requests the interpretation report server 7 to store the interpretation report. The interpretation report server 7 prepares the interpretation report and the medical image 2 in a predetermined format, and stores the interpretation report and the medical image in association with each other in the interpretation report database 8.

At time t4 of FIG. 3, in a case where the interpretation of the medical image 2 is ended, since the display window of the medical image 2 for which the interpretation is ended is switched to the display window of the medical image 1, step ST13 is positive. The management unit 33 of the terminal device 3 notifies the warning server 10 that the display window of the medical image 2 is closed and that the display window of the medical image 1 is in the active state (step ST14). In a case where the notification is completed, it is determined in step ST15 that the interpretation is ended, and the processing returns to step ST13 and is in the waiting state until the switching of the next image or the switching of the program occurs until the interpretation support program is ended.

In a case where the notification is received from the terminal device 3, the determination of step ST30 is positive, and the execution program management unit 13 of the warning server 10 records the received state of the program in the state transition table as described above (step ST31). The detection unit 14 of the warning server 10 determines whether the interpretation of the medical image 1 is interrupted or the interpretation is resumed based on the information of the state transition table (step ST32). In a case where it is determined that the display window of the medical image 1 is active and the interpretation of the medical image 1 is to be resumed (step ST33 is negative and step ST36 is positive), the counter C1 of the medical image 1 is reset (Step ST37). Meanwhile, since the display window of the medical image 2 is already closed, it is not determined that the interpretation is interrupted or the interpretation is resumed. In a case where the determination of the interruption of the interpretation or the resumption of the interpretation is completed for all the active medical images and the inactive medical images being interpreted, the processing returns from step ST35 to step ST30, and is in the waiting state until the next notification is received from the terminal device 3.

Next, a case where a program other than the interpretation support program is activated will be described. Here, a case where another program is, for example, a conference support program for performing a conference in a chat format will be described.

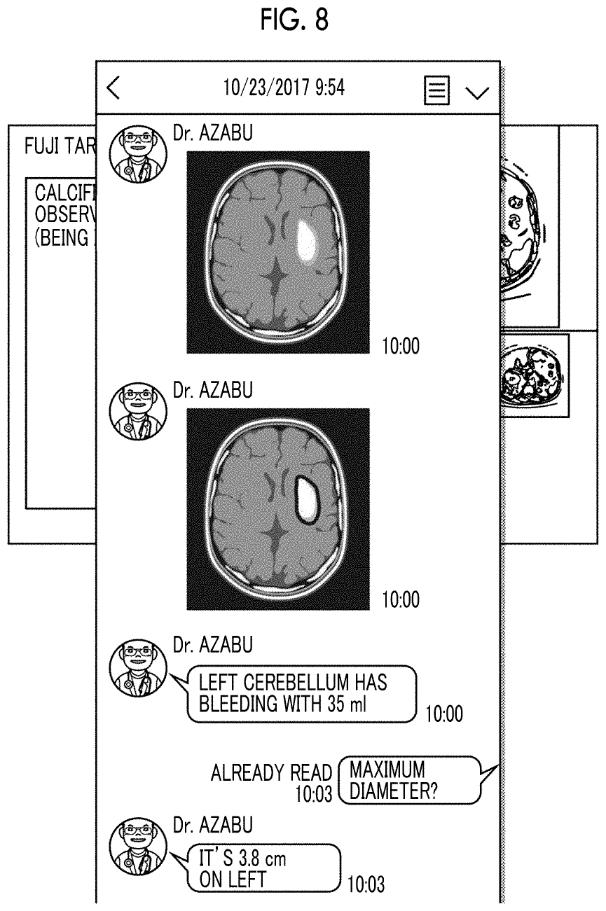

For example, while the medical image 1 is continuously being interpreted, in a case where the doctor of the clinical department transmits an invitation to the conference for another patient from the clinical department WS 4 to the terminal device 3, the conference support program is activated on the terminal device 3 (time t5 of FIG. 3), and a message indicating that the user is invited to the conference is displayed on the terminal device 3. In a case where the invitation message is approved on the terminal device 3, for example, a chat screen of the conference support program is displayed as shown in FIG. 8. The display window for chat is active, and the display window for the medical image 1 is inactive. FIG. 8 shows an example in which a conference is held with “Dr. Azabu”, for example. Since the interpretation support program P is switched to the conference support program, the determination of step ST13 is positive. The management unit 33 of the terminal device 3 notifies the warning server 10 that the conference support program is in execution, that the display window for chat is in the active state, and that the display window of the medical image 1 is in the inactive state (step ST14). In a case where the notification is completed, it is determined in step ST15 that the interpretation is ended. The processing returns to step ST13, and is in the waiting state until the switching from the conference support program to the interpretation support program occurs.

In a case where the notification is received from the terminal device 3, the determination of step ST30 is positive, and the execution program management unit 13 of the warning server 10 records the received state of the interpretation support program in the state transition table (step ST31). As described above, the detection unit 14 of the warning server 10 determines whether the interpretation of the medical image is interrupted in step ST33 and determines whether or not to resume (or start) the interpretation of the medical image in step ST36 based on the information of the state transition table. Since the display window of the medical image 1 is inactive and it is determined that the interpretation of the medical image 1 is interrupted, step ST33 is positive, and the counter C2 of the medical image 1 starts counting from time t5 of FIG. 3. Since there is no other medical image being interpreted, it is determined that the interruption of the interpretation and the resumption of the interpretation is ended, and step ST35 is positive. The processing returns to step ST30.

In a case where the user ends the conference support program of the terminal device 3 before the counter C2 counts up (t6 of FIG. 3), and in a case where the user switches from the conference support program to the interpretation support program P again, step ST13 is positive. The display window for chat is closed, the medical image 1 is displayed, and the interpretation report which is the operation input can be input. The management unit 33 of the terminal device 3 notifies the warning server 10 that the conference support program is ended, that the display window for chat is closed, and that the display window for the medical image 1 is in the active state (step ST14).

In a case where the notification is received from the terminal device 3, step ST30 is positive, and the execution program management unit 13 of the warning server 10 records the received state of the interpretation support program in the state transition table (step ST31). The detection unit 14 of the warning server 10 determines whether the interpretation of the medical image 1 is interrupted or the interpretation is resumed (or started) based on the information of the state transition table (step ST32). In a case where the display window of the medical image 1 is active and it is determined that the interpretation of the medical image 1 is to be resumed, step ST33 is negative, step ST36 is positive, and the counter C2 of the medical image 1 is reset (step ST37). Since the image being interpreted is only the medical image 1, it is determined that the determination of the interruption of the interpretation or the resumption of the interpretation is completed. The processing returns from the step ST35 to the step ST30, and is in the waiting state until the next notification is received from the terminal device 3.

In a case where the user performs an operation of ending the interpretation of the medical image 1 on the terminal device 3, step ST15 is positive. The interpretation support program P requests the interpretation report server 7 to store the interpretation report, and ends the interpretation support program P (step ST16). The interpretation report server 7 prepares the interpretation report and the medical image 1 in a predetermined format, and stores the interpretation report and the medical image in association with each other in the interpretation report database 8. The terminal device 3 notifies the warning server 10 of the end of the interpretation support program P (step ST17).

Next, a second embodiment will be described. Although it has been described in the first embodiment that the warning device (warning server) and the terminal device are connected via the network and the state of the program of the terminal device is managed by the warning server, a case where the warning device manages the state transition of the program installed on the host device and displays the warning will be described in the second embodiment. The same components as those of the first embodiment are denoted by the same references, and detailed description is omitted.

Since the overall configuration of the medical support system is the same as that of the first embodiment shown in FIG. 1, detailed description is omitted. In the present embodiment, the warning program is installed on the interpretation WS 3, and thus, the interpretation WS function as the warning device. Hereinafter, the interpretation WS 3 will be described as the warning device 3.

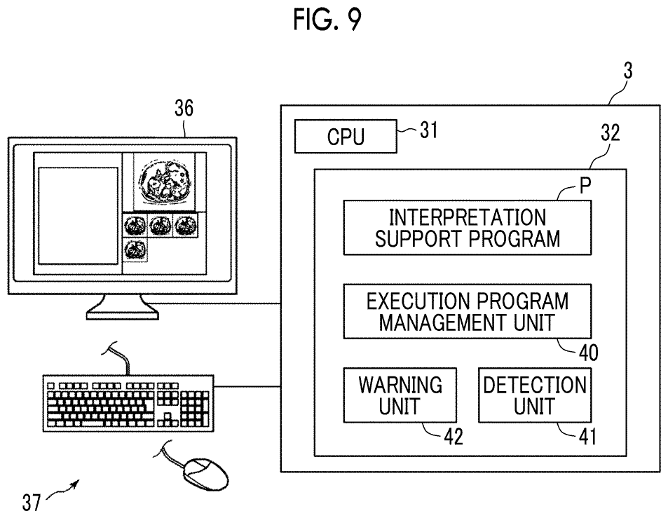

As shown in FIG. 9, the warning device 3 according to the second embodiment functions as an execution program management unit 40, a detection unit 41, and a warning unit 42.

The second embodiment has substantially the same function as the execution program management unit 13 of the first embodiment in which the execution program management unit 40 records the information on the state of each program in the state transition table for all the programs installed on the warning device 3 and manages the state of each program. However, the second embodiment is different from the first embodiment in that the state of the program installed on the warning device 3 is managed by the host computer instead of managing the state of the program in the separately provided server as in the first embodiment.

The detection unit 41 executes the interpretation support program P for interpreting the medical image, and detects that the operation of interrupting the interpretation is performed in a case where the operation input such as the interpretation report is executable. Alternatively, the interpretation support program P detects that the operation of resuming the interpretation from the state in which the interpretation is interrupted is performed. Since the second embodiment is the same as the first embodiment in that the interruption of the interpretation and the resumption of the interpretation are determined based on the state transition table, the detailed description is omitted.

The warning unit 42 issues the warning in a case where the operation of resuming the interrupted interpretation is not detected within a predetermined time from the time at which the operation of interrupting the interpretation is detected. Specifically, the warning is performed by displaying a message on a screen of the display 36. Alternatively, a mark indicating the warning may be displayed on the screen, or a warning sound may be sounded.

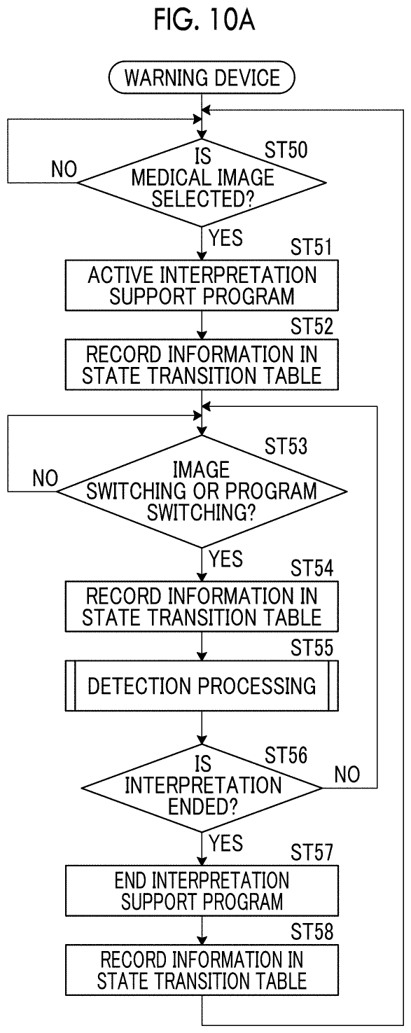

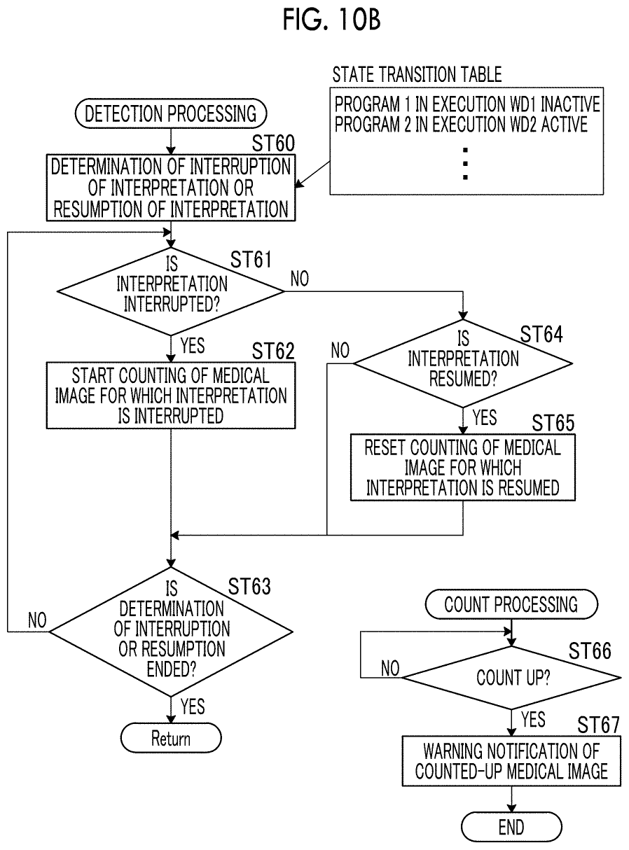

Next, a method of detecting the operations for interrupting the interpretation and resuming the interpretation by using the example of the display screen of FIGS. 5 to 8 according to a state transition diagram of the program of FIG. 3 and flowcharts of FIGS. 10A and 10B will be described. FIG. 10A shows a flow of main processing of the entire warning device, and FIG. 10B shows ae flow of detection processing. Since the display screens of FIGS. 5 to 8 are the same as those of the first embodiment, detailed description of the display screens is omitted. First, a case in which the interpretation of the image being interpreted is interrupted and the interpretation of another image is started on the interpretation support program will be described.

The interpretation target list is displayed on the warning device 3. In a case where the user selects the medical image 1 to be interpreted, step ST50 is positive, and the interpretation support program P is activated (time t1 of FIG. 3, step ST51). The interpretation support program P acquires the selected medical image 1 to be interpreted from the image server 5, and displays the medical image 1 on the display 36 as shown in FIG. 5. The execution program management unit 40 records information indicating that the interpretation support program P for the medical image 1 is in execution and information indicating that the display window of the medical image 1 is in the active state in the state transition table (step ST52).

In a case where the user selects the urgent case from the interpretation target list during the interpretation of the medical image 1, since the interpretation support program P acquires the selected medical image 2 to be interpreted from the image server 5, switches from the medical image 1 to the medical image 2, and displays the switched medical image on the display 36 as shown in FIG. 6, step ST53 is positive. The execution program management unit 40 records information indicating that the interpretation support program P for the medical image 2 is in execution, information indicating that the display window of the medical image 2 is in the active state, and information indicating that the display window of the medical image 1 is in the inactive state in the state transition table (step ST54). The number of the image active and the number of the image inactive for the interpretation support program are stored and the counter for the active image and the counter for the inactive image are also stored in the state transition table.

Subsequently, the detection processing is executed (step ST55), and the interruption of the interpretation or the resumption of the interpretation is determined by the detection unit 41 (step ST60). From time t2 of FIG. 3, the display window of the medical image 2 is active, and the display window of the medical image 1 is inactive as shown in FIG. 6. Since it is determined that the interpretation of the medical image 1 is interrupted, step ST61 is positive, and the counter C1 of the medical image 1 starts counting from time t2 (step ST62). The processing returns from step ST63 to step ST61, and the determination of whether the interpretation of the medical image 2 is interrupted or the interpretation is resumed is performed. Since the interpretation of the medical image 2 is started, the determination of step ST61 is negative, the determination of step ST64 is positive, and the counter for the medical image 2 is maintained in the reset state (step ST65). In a case where the determination of the interruption of the interpretation or the resumption of the interpretation is completed for all of the active medical image and the inactive medical image being interpreted, the determination of step ST63 is positive, and the processing returns to the main processing (step ST55). In step ST56, it is determined that the interpretation is ended. The processing returns to step ST53 until the interpretation using the interpretation support program is ended, and is in the waiting state until the switching of the next image or the switching of the program occurs.

Meanwhile, the counter C1 which starts counting is periodically checked by the count processing until the counter counts up (step ST66). In a case where a predetermined time elapses after the interpretation of the medical image 1 is interrupted, that is, the medical image is inactive (time t3 of FIG. 3), since the counter Cl counts up, step ST66 is positive, and the warning unit 42 displays the warning as shown in FIG. 7 (step ST67).

In a case where the interpretation of the medical image 2 is ended at time t4 of FIG. 3, the display window of the medical image 2 is closed and is switched to the display window of the medical image 1, and step ST53 is positive. The execution program management unit 40 records information indicating that the display window for the medical image 2 is closed, and information indicating that the display window of the medical image 1 is in the active state in the state transition table (step ST54).

Subsequently, the processing proceeds to the detection processing (step ST55), and the detection unit 14 determines whether the interpretation of the medical image 1 is interrupted or the interpretation is resumed based on the information of the state transition table (step ST60). The display window of the medical image 1 is active, it is determined that the interpretation of the medical image 1 is to be resumed (step ST61 is negative and step ST64 is positive), and the counter C1 of the medical image 1 is reset (step ST65). Meanwhile, since the display window of the medical image 2 is already closed, it is not determined that the interpretation is interrupted or the interpretation is resumed. In a case where the determination of the interruption of the interpretation or the resumption of the interpretation is ended, step ST63 is positive, and the processing returns to the main processing (step ST55). It is determined that the interpretation is not ended in step ST56, and the processing returns to step ST53, and is in the waiting state until the switching of the next image or the switching of the program occurs (step ST53).

Next, a case where a program other than the interpretation support program is activated will be described. Similarly to the first embodiment, a case where another program is, for example, the conference support program for performing the conference in the chat format will be described.

For example, while the medical image 1 is being interpreted on the warning device 3, in a case where the doctor of the clinical department transmits a request to participate in the conference for another patient from the clinical department WS 4 to the warning device 3 (interpretation WS 3), the conference support program is activated on the warning device 3 (time t5 of FIG. 3), and a message indicating that the user is invited to the conference is displayed. In a case where the invitation message is approved by the warning device 3, a conference chat screen is displayed as shown in FIG. 8. The display window for chat is active, and the display window for the medical image 1 is inactive. Since the interpretation support program P is switched to the conference support program, step ST53 is positive, and the execution program management unit 40 records information indicating that the conference support program is in execution, information indicating that the display window for chat is in the active state, and information indicating that the display window of the medical image 1 is in the inactive state in the state transition table (step ST54).

Subsequently, the processing proceeds to the detection processing (step ST55), and the detection unit 41 determines whether the interpretation of the medical image 1 is interrupted or the interpretation is resumed based on the information in the state transition table (step ST60). Since the display window of the medical image 1 is inactive, it is determined that the interpretation is interrupted. Step ST61 is positive, and the counter C2 of the medical image 1 starts counting from time t5 of FIG. 3 (step ST62). Since there is no image being interpreted other than the medical image 1, it is determined that the determination of the interruption of the interpretation and the resumption of the interpretation is ended, and step ST63 is positive. The processing returns to the main processing (step ST55). In step ST56, it is determined that the interpretation is not ended, and the processing returns to step ST53 and is in the waiting state until the switching of the next image or the switching of the program occurs (step ST53).

In a case where the user ends the conference support program of the terminal device 3 before the counter C2 counts up (t6 of FIG. 3), the conference support program is switched to the interpretation support program P again without displaying the warning, and step ST53 is positive. The display window for chat is closed, and the medical image 1 is displayed. The execution program management unit 40 records information indicating that the conference support program is ended, information indicating that the display window for chat is closed, and information indicating that the display window of the medical image 1 is in the active state in the state transition table (step ST54).

Subsequently, the detection processing is executed (step ST55), and the detection unit 41 determines whether the interpretation of the medical image 1 is interrupted or the interpretation is resumed based on the information of the state transition table (step ST60). It is determined that the interpretation of the medical image 1 is to be resumed (step ST61 is negative and step ST64 is positive), and the counter C2 of the medical image 1 is reset (step ST65). In a case where the user performs an operation of selecting the end of the interpretation of the medical image 1, step ST56 is positive. The interpretation support program P requests the interpretation report server 7 to store the interpretation report, and the interpretation support program P is ended (step ST57). The interpretation report server 7 prepares the interpretation report and the medical image 1 in a predetermined format, and stores the interpretation report and the medical image in association with each other in the interpretation report database 8. The execution program management unit 40 records the end of the interpretation support program P in the state transition table (step ST58).

As described above in detail in the first embodiment and the second embodiment, even in a case where the interpretation of another patient is requested in the middle of the interpretation or the doctor participates in the conference using the terminal device by detecting that the interpretation is interrupted and by issuing the warning in a case where a predetermined time or more elapses, the interpretation can be resumed without neglecting the patient whose interpretation is interrupted.

In the aforementioned embodiment, a hardware structure of a processing unit that executes various processing such as a diagnosis support device includes various processors to be described below. Various processors include a Central Processing Unit (CPU) which is a general-purpose process functioning as various processing units by executing the software (program), a Programmable Logic Device (PLD) which is a processor of changing a circuit configuration after Field Programmable Gate Array (FPGA) is manufactured, and a dedicated electric circuit which is a processor having a circuit configuration specifically designed to execute specific processing such an Application Specific Integrated Circuit (ASIC).

One processing unit may be constituted by one of these various processors, or may be a combination of two or more processors of the same type or different types (for example, a combination of a plurality of FPGAs or a combination of a CPU and an FPGA). A plurality of processing units may be constituted by one processor. As an example in which the plurality of processing units is constituted by one processor, first, one processor is constituted by a combination of one or more CPUs and software and this processor functions as the plurality of processing units as represented by a computer such as a client or a server. Second, a processor that realizes the functions of the entire system including the plurality of processing units by using one Integrated Circuit (IC) chip is used as represented by a System On Chip (SoC). As described above, the various processing units are constituted by using one or more of the aforementioned various processors as a hardware structure.

### EXPLANATION OF REFERENCES

1: medical information system

2: modality

3: interpretation workstation

4: clinical department workstation

5: image server

6: image database

7: interpretation report server

8: interpretation report database

9: network

10: warning device

11, 31: CPU

12: memory

13: execution program management unit

14: detection unit

15: warning unit

30: terminal device

32: memory

33: management unit

34: notification unit

35: warning notification unit

36: display

37: input device

40: execution program management unit

41: detection unit

42: warning unit

C1: counter

C2: counter

P: interpretation support program

What is claimed is:

1. A warning device comprising:
a detection unit that detects an operation of interrupting interpretation in a state in which an operation input is executable during execution of an interpretation support program for supporting interpretation of a medical image; and
a warning unit that issues a warning in a case where an operation of resuming the interrupted interpretation is not detected within a predetermined time from a time at which the interruption operation is detected.

2. The warning device according to claim 1,
wherein the interruption operation is an operation of starting execution of another program other than the interpretation support program.

3. The warning device according to claim 2,
wherein the other program is a program for transmitting and receiving a message related to a conference of the medical image to and from another terminal device.

4. The warning device according to claim 1,
wherein the interruption operation is an operation of switching an image to a medical image other than the medical image being interpreted.

5. The warning device according to claim 4,
wherein the operation of switching the image is an operation of switching the image to the medical image for which the interpretation is interrupted during execution of interpretation of a predetermined medical image or an operation of switching the image to a new medical image for which the interpretation is started during the execution of the interpretation of the predetermined medical image.

6. A warning method performed by a warning device, comprising:
a detection step of detecting an operation of interrupting interpretation in a state in which an operation input is executable during execution of an interpretation support program for supporting interpretation of a medical image; and
a warning step of issuing a warning in a case where an operation of resuming the interrupted interpretation within a predetermined time from a time at which the interruption operation is not detected.

7. A non-transitory computer readable recording medium storing a warning program causing a computer to execute:
a detection step of detecting an operation of interrupting interpretation in a state in which an operation input is executable during execution of an interpretation support program for supporting interpretation of a medical image; and
a warning step of issuing a warning in a case where an operation of resuming the interrupted interpretation within a predetermined time from a time at which the interruption operation is not detected.

8. A warning device comprising:
a memory that stores a command to be executed by a computer; and
a processor configured to execute the stored command,
wherein the processor detects an operation of interrupting interpretation in a state in which an operation input is executable during execution of an interpretation support program for supporting interpretation of a medical image, and
warns a warning in a case where an operation of resuming the interrupted interpretation is not detected within a predetermined time from a time at which the interruption operation is detected.
